SEQUENCE LISTING (8 sequences)

SEQ ID NO: 1 - DNA, 442 bp, synthetic construct
  source 1..442: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttctctggctaactagagaacccactgcttactggcttatcgaaattaatacgactcactatagggagtcccaagctggctagcgccaccatgg

SEQ ID NO: 2 - DNA, 428 bp, synthetic construct
  source 1..428: mol_type = other DNA
tccctatcagtgatagagaaacgcctccctatcagtgatagagaaaagcttccctatcagtgatagagaagaagaggtgtattccctatcagtgatagagaaacgcctccctatcagtgatagagaaaagcttccctatcagtgatagagaagatgtgaggtattccctatcagtgatagagaaacgcctccctatcagtgatagagaaaagcttccctatcagtgatagagaataggcctagtattccctatcagtgatagagaaacgcctccctatcagtgatagagaaaagcttccctatcagtgatagagaaacgctctctagacaacaaaatgtcgtaacaagggcggtaggcgtgtacggtgggaggtctatataagcagagctcgcagtgaaccgtcagatccagagctagcgccaccatg

SEQ ID NO: 3 - DNA, 385 bp, synthetic construct
  source 1..385: mol_type = other DNA
tccctatcagtgatagagaaacgcctccctatcagtgatagagaaaagcttccctatcagtgatagagaagaagaggtgtattccctatcagtgatagagaaacgcctccctatcagtgatagagaaaagcttccctatcagtgatagagaagatgtgaggtattccctatcagtgatagagaaacgcctccctatcagtgatagagaaaagcttccctatcagtgatagagaataggcctagtattccctatcagtgatagagaaacgcctccctatcagtgatagagaaaagcttccctatcagtgatagagaaacgctctctagagggtatataatgggggccactagtctactaccagagctcatcgctagcgccaccatg

SEQ ID NO: 4 - DNA, 464 bp, synthetic construct
  source 1..464: mol_type = other DNA
tccctatcagtgatagagaaacgcctccctatcagtgatagagaaaagcttccctatcagtgatagagaagaagaggtgtattccctatcagtgatagagaaacgcctccctatcagtgatagagaaaagcttccctatcagtgatagagaagatgtgaggtattccctatcagtgatagagaaacgcctccctatcagtgatagagaaaagcttccctatcagtgatagagaataggcctagtattccctatcagtgatagagaaacgcctccctatcagtgatagagaaaagcttccctatcagtgatagagaaacgctctctagaggtaggcgtgtacggtgggaggtctatataagcagagctgagctctctggctaactagagaacccactgcttactggcttatcgaaattaatacgactcactatagggagtccccaagctggctagcgccaccatg

SEQ ID NO: 5 - DNA, 526 bp, synthetic construct
  source 1..526: mol_type = other DNA
tccctatcagtgatagagaaacgcctccctatcagtgatagagaaaagcttccctatcagtgatagagaagaagaggtgtattccctatcagtgatagagaaacgcctccctatcagtgatagagaaaagcttccctatcagtgatagagaagatgtgaggtattccctatcagtgatagagaaacgcctccctatcagtgatagagaaaagcttccctatcagtgatagagaataggcctagtattccctatcagtgatagagaaacgcctccctatcagtgatagagaaaagcttccctatcagtgatagagaaacgctctctagaacgtatgtcgaggtaggcgtgtacggtgggcgcctataaaagcagagctcgtttagtgaaccgtcagatcgcctggagcaattccacaacacttttgtcttatacttctctggctaactagagaacccactgcttactggcttatcgaaattaatacgactcactatagggagtcccaagctggctagcgccaccatg

SEQ ID NO: 6 - skipped

SEQ ID NO: 7 - DNA, 322 bp, synthetic construct
  source 1..322: mol_type = other DNA
tccctatcagtgatagagaaacgcctccctatcagtgatagagaaaagcttccctatcagtgatagagaagaagaggtgtattccctatcagtgatagagaaacgcctccctatcagtgatagagaaaagcttccctatcagtgatagagaagatgtgaggtattccctatcagtgatagagaaacgcctccctatcagtgatagagaaaagcttccctatcagtgatagagaataggcctagtattccctatcagtgatagagaaacgcctccctatcagtgatagagaaaagcttccctatcagtgatagagaaacgctc

SEQ ID NO: 8 - DNA, 25 bp, synthetic construct
  source 1..25: mol_type = other DNA
tctagagggtatataatgggggcca

(19) United States
(12) Patent Application Publication
(10) Pub. No.: US 2026/0002173 A1
(43) Pub. Date: Jan. 1, 2026

(54) DOXYCYCLINE INDUCIBLE EXPRESSION SYSTEM

(71) Applicant: Northwestern University, Evanston, IL (US)

(72) Inventors: Joshua N. Leonard, Wilmette, IL (US); Hailey Ilyse Edelstein, Evanston, IL (US)

(73) Assignee: Northwestern University, Evanston, IL (US)

(21) Appl. No.: 19/248,944

(22) Filed: Jun. 25, 2025

Related U.S. Application Data
(60) Provisional application No. 63/664,404, filed on Jun. 26, 2024.

(51) Int. Cl. C12N 15/85

(52) U.S. Cl. CPC C12N 15/85; C12N 2800/107; C12N 2830/003

(57) ABSTRACT

The present disclosure provides, among other things, novel tetracycline-inducible expression systems and methods for controlling expression of a target gene in a mammalian cell.

### CROSS-REFERENCE TO RELATED APPLICATIONS

The present application claims priority to U.S. Provisional Application No. 63/664,404, filed Jun. 26, 2024, the entire contents of which are incorporated herein by reference.

### GOVERNMENT LICENSING RIGHTS STATEMENT

This invention was made with government support under grant number EB026510 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 11, 2025, is named 121384-0277_SL.xml and is 9,834 bytes in size.

### BACKGROUND

Tetracycline-inducible, such as doxycycline (DOX)-inducible, gene expression systems comprise a transcriptional activator (e.g., an engineered transcriptional activator) and a tetracycline-inducible promoter to which the transcriptional activator binds to induce transcription of a target gene. Typically, the transcriptional activator (e.g., engineered transcriptional activator) is a variant of the bacterial tetracycline repressor protein, reverse tetracycline transactivator (rtTA) or a variant thereof, which can bind to a tetracycline operator (TetO) site within the tetracycline-inducible promoter only when rtTA is bound to a tetracycline (e.g., DOX), thereby promoting expression of a target gene operably linked to the tetracycline-inducible promoter. rtTA is understood not to bind to TetO when in the absence of (e.g., when rtTA is unbound) to a tetracycline.

A canonical tetracycline-inducible promoter comprises seven TetO sites spaced 17 base pairs apart and located upstream (5′ on the nucleic acid) of a minimal promoter. Together, the transcriptional activator and tetracycline-inducible promoter form a tetracycline-inducible gene expression system that drives target gene expression in mammalian cells when cultured with a tetracycline in the cell culture media. This type of gene expression system is particularly valuable for activating expression of genes stably integrated in the genome of mammalian cells, with applications spanning fundamental research to biomanufacturing.

However, a continued challenge with such tetracycline-inducible gene expression systems is “leaky” background expression (e.g., tetracycline-inducible promoter activity in the absence of a tetracycline) and/or a limited upper bound of induced expression of the target gene (e.g., limited tetracycline-inducible promoter activity in the presence of a tetracycline). Thus, there remains a continued need for improved tetracycline-inducible expression systems with (i) reduced background expression of target genes (e.g., expression in the absence of a tetracycline); (ii) increased expression of target genes (e.g., in the presence of a tetracycline); and/or (iii) increased fold induction of target gene expression (increased ratio of induced target gene expression relative to background expression).

### SUMMARY OF THE INVENTION

In one aspect, the present disclosure provides a method for controlling expression of a target gene in a mammalian cell, comprising: (a) transiently transfecting or stably genomically integrating a mammalian cell with (1) a first nucleic acid comprising a nucleotide sequence comprising a tetracycline-inducible promoter that controls expression of the target gene; and (2) a second nucleic acid comprising a nucleotide sequence encoding a transcriptional activator that binds to TetO sites, thereby producing an engineered mammalian cell; and (b) contacting the engineered mammalian cell with a tetracycline to induce expression of the target gene, wherein the tetracycline-inducible promoter comprises a minimal promoter and a TetO site array in which TetO sites are spaced more compactly and with more TetO sites than in pTRE3GV. In one aspect, the present disclosure provides a vector comprising a minimal promoter and a TetO site array in which TetO sites are spaced more compactly and with more TetO sites than in pTRE3GV.

### BRIEF DESCRIPTION OF THE DRAWINGS

FIG. 1A provides a schematic of pTRE3GV dox-inducible gene expression system. This Tet ON gene expression system employs the reverse tetracycline transactivator (rtTA), a fusion protein containing a transcriptional activation domain (AD) and a mutated form of the bacterial tetracycline repressor protein (rTetR)l. In the absence of tetracycline or analogs such as doxycycline (dox), rtTA cannot bind to its cognate DNA operator sequence. In the presence of dox, rtTA undergoes a conformational change that enables binding to its DNA operator sequence, subsequent recruitment of transcriptional machinery via its AD to the minimal promoter and resulting promoter activation and transgene expression. This dox-inducible promoter, the pTRE3GV promoter, contains 7 widely spaced operator sites upstream of a minimal CMV promoter. FIG. 1B provides the promoter sequence of the TRE3GV promoter (SEQ ID NO: 1).

FIG. 2A provides a schematic of x12TetO CMV53 dox-inducible gene expression system. This dox-inducible expression system uses the same rtTA protein and mechanism described in FIG. 1A. This dox-inducible promoter contains 12 compactly spaced operator sites upstream of a minimal CMV promoter and initiator region (previously named CMV53). FIG. 2B provides the promoter sequence of x12TetO CMV53 promoter (SEQ ID NO: 2).

FIG. 3A provides a schematic of x12TetO YB_TATA dox-inducible gene expression system. This dox-inducible expression system uses the same rtTA protein and mechanism described in FIG. 1A. This dox-inducible promoter contains 12 compactly spaced operator sites upstream of a synthetic YB_TATA promoter. FIG. 3B provides the promoter sequence of the x12TetO YB_TATA promoter (SEQ ID NO: 3).

FIG. 4A provides a schematic of x12TetO minCMV dox-inducible gene expression system. This dox-inducible expression system uses the same rtTA protein and mechanism described in FIG. 1A. This dox-inducible promoter contains 12 compactly spaced operator sites upstream of a minimal CMV promoter. FIG. 4B provides the promoter sequence of the x12TetO minCMV promoter (SEQ ID NO: 4).

FIG. 5A provides a schematic of x12TetO minCMVT dox-inducible gene expression system. This dox-inducible expression system uses the same rtTA protein and mechanism described in FIG. 1A. This dox-inducible promoter contains 12 compactly spaced operator sites upstream of a the same minimal CMV promoter employed in the pTRE3GV system (referred to as minCMVT) depicted in FIG. 1A. FIG. 5B provides the promoter sequence of the x12TetO minCMVT promoter (SEQ ID NO: 5).

FIG. 6A-6C provide results evaluating various x12TetO promoters compared to pTRE3GV. FIG. 6A provides a schematic of transiently transfected dox-inducible gene expression systems. HEK293FT cells were transfected with three plasmids for gene expression: a transfection control plasmid that encodes constitutive expression via the human cytomegalovirus (CMV) promoter of mTagBFP2 to identify transfected (blue fluorescence positive) cells; a plasmid that encodes constitutive expression via the human elongation factor-1 alpha (hEF1a) promoter of rtTA; a plasmid that encodes dox-inducible expression of the reporter gene DsRedExpress2 to evaluate promoter activity when cells are treated with dox. FIG. 6B provides results from reporter expression of each dox-inducible promoter as a function of dox dose. Data shown represent the mean fluorescence intensity (in absolute units of molecules of equivalent PE-TexasRed, MEPTRs) of transfected, single cells. Transfected cells were identified as expressing mTagBFP2. Each data point represents the mean across three biologic replicates and error bars depict the standard error of the mean. FIG. 6C provides background reporter expression of the promoter/reporter alone with no rtTA, background reporter expression of the promoter/reporter and rtTA treated with 0 ng/ml dox, and induced reporter expression of the promoter/reporter and rtTA treated with 1,000 ng/ml dox for each dox-inducible promoter. Data shown represent mean fluorescence intensity of transfected, single cells and is depicted on a logarithmic scale. Each bar represents the mean across three biologic replicates and error bars depict the standard error of the mean.

FIG. 7A-7K shows construction of a low background doxycycline-inducible promoter with high fold induction. FIG. 7A provides a schematic of the Tet ON gene expression system, which employs the reverse tetracycline transactivator (rtTA), a fusion protein containing a transcriptional activation domain (AD) and a mutated form of the bacterial tetracycline repressor protein (rTetR). In the absence of tetracycline or analogs such as doxycycline (dox), rtTA cannot bind to its cognate DNA operator sequence. In the presence of dox, rtTA undergoes a conformational change that enables binding to its DNA operator sequence, subsequent recruitment of transcriptional machinery via its AD to the minimal promoter and resulting promoter activation and transgene expression. One challenge of the original Tet ON gene expression system is the leaky background gene expression in the absence of dox. To enhance this system, a reported TetO site array was paired with more TetO sites spaced more compactly with a panel of minimal promoters with different levels of basal activity. FIG. 7B provides a schematic of landing pad integration vector cargo. Briefly, HEK293FT-LP cells were transfected with the integration vector and a plasmid encoding expression of Bxb1 recombinase and selected for those that were resistant to both puromycin and blasticidin. Selected cells contained one of the tested dox-inducible promoters regulating expression of an mNeonGreen reporter gene to evaluate promoter activity when cells were treated with dox. FIG. 7C shows reporter expression of each dox-inducible promoter when implemented in the landing pad as a function of dox dose. FIG. 7D shows histograms visualizing distribution of reporter expression across treated cells that underly the bulk data shown in FIG. 7C. Exceptionally tight off state of x12TetO YB_TATA (low background expression when treated with 0 ng/mL dox compared to all other tested promoters) was evident. FIG. 7E shows background reporter expression when treated with 0 ng/ml dox and induced reporter expression when treated with 1,000 ng/mL dox for each dox-inducible promoter. Background expression of DoxMax (x12TetO YB_TATA) was significantly lower than the other promoter designs (single-factor ANOVA, ***p<0.001). The exceptionally low background of DoxMax facilitates characterization of synTFs in this study by widening the lower end of the range of accessible synTF expression levels in the genome. Additionally, low background has advantages for toxicity and burden caused by even low levels of expression of very strong synTFs. The maximal induced reporter expression when treated with 1,000 ng/mL dox is significantly different between all promoter designs (single-factor ANOVA, p<0.05). Data shown in (FIG. 7C-7E) represent the fluorescence intensity or mean fluorescence intensity of miRFP720-expressing, single cells. Error bars depict the standard error of the mean (S.E.M.). FIG. 7F shows fold induction of each dox-inducible promoter as a function of dox dose. Data shown represent the fold induction (reporter expression at dose of dox on x-axis divided by the reporter expression in the sample treated with 0 ng/mL dox) of miRFP720-expressing single cells. Each data point represents the mean across three biologic replicates of miRFP720-expressing, single cells and error bars depict the standard error of the mean. FIG. 7G shows a schematic of all-in-one lentiviral vector for the x12TetO YB_TATA-based dox-inducible gene expression system (DoxMax). Briefly, HEK293FT cells were engineered to stably express the cargo depicted here using the depicted lentiviral vector. This workflow involves transducing cells with lentivirus and selecting for cells that are resistant to puromycin. FIG. 7H shows reporter expression of selected cells with a single or multiple copies of this system as a function of dox dose. FIG. 7I shows background reporter expression when treated with 0 ng/ml dox and induced reporter expression when treated with 1,000 ng/mL dox. Background reporter expression was not different between populations with single and multicopy integrations of the lentiviral reporter construct and unmodified HEK293FT cells (multi-factor ANOVA, not significant, n.s. p>0.05), suggesting that background expression was still low even when implemented in multiple copies. Although background did not increase with additional copies, induced reporter output at all doxycycline doses greater than 1 ng/mL was significantly increased in cells with multiple copies of the lentiviral reporter construct compared to cells with a single copy (multi-factor ANOVA, **p<0.01). FIG. 7J provides histograms visualizing distribution of reporter expression across treated cells that underly the bulk data shown in FIG. 7H. Data shown in FIG. 7H-7J represent fluorescence intensity or mean fluorescence intensity of mNeonGreen-expressing single cells. Each bar represents the mean across three biologic replicates and error bars depict the standard error of the mean (S.E.M.). FIG. 7K shows fold induction as a function of dox dose. Data shown represent the fold induction (reporter expression at dose of dox on x-axis divided by the reporter expression in the sample treated with 0 ng/mL dox) of mNeonGreen-expressing single cells. Each data point represents the mean across three biologic replicates and error bars depict the standard error of the mean (S.E.M.). Abbreviations: rTetR, reverse tetracycline repressor; AD, activation domain; rtTA, reverse tetracycline transactivator; dox, doxycycline; tet, tetracycline; TetO, tetracycline repressor operator; CMV, cytomegalovirus; pCMV, cytomegalovirus promoter; pCAG, cytomegalovirus enhancer with chicken beta-actin promoter; miRFP720, monomeric infrared fluorescent protein 720; pEF1a, human elongation factor 1 alpha promoter; BlastR, blasticidin resistance gene; PuroR, puromycin resistance gene; pTet, tetracycline-inducible promoter; AAVS1, adeno-associated virus integration site 1; cHS4, chicken hypersensitive site-4 insulator; MEFLs, molecules of equivalent fluorescein; MEPTRs, molecules of equivalent PE-TexasRed; IRES, internal ribosomal entry site; LTR, long terminal repeat; WPRE, woodchuck hepatitis virus posttranscriptional regulatory element; n.s., not significant; FI, fluorescence intensity; LTR ΔU3, LTR with truncated U3 region.

### DETAILED DESCRIPTION

It is to be appreciated that certain aspects, modes, embodiments, variations, and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

Inducible gene expression systems are important tools for the advancement of biological (e.g., biomedical) research and related applications. For example, tetracycline-inducible, such as doxycycline (DOX)-inducible, gene expression systems comprise a transcriptional activator (e.g., an engineered transcriptional activator) and a tetracycline-inducible promoter to which the transcriptional activator binds to induce transcription of a target gene. Typically, the transcriptional activator (e.g., engineered transcriptional activator) is a variant of the bacterial tetracycline repressor protein, reverse tetracycline transactivator (rtTA) or a variant thereof, which can bind to the tetracycline operator (TetO) site only when rtTA is bound to a tetracycline (e.g., DOX), thereby promoting expression of a target gene operably linked to the tetracycline-inducible promoter. rtTA is understood not to bind to TetO when in the absence of (e.g., when rtTA is unbound) to a tetracycline.

A canonical tetracycline-inducible promoter comprises seven TetO sites spaced 17 base pairs apart and located upstream (5′ on the nucleic acid) of a minimal promoter. Together, the transcriptional activator and tetracycline-inducible promoter form a tetracycline-inducible gene expression system that drives target gene expression in mammalian cells when cultured with a tetracycline in the cell culture media. This type of gene expression system is particularly valuable for activating expression of genes stably integrated in the genome of mammalian cells, with applications spanning fundamental research to biomanufacturing.

However, a continued challenge with such tetracycline-inducible gene expression systems is “leaky” background expression (e.g., tetracycline-inducible promoter activity in the absence of a tetracycline) and/or a limited upper bound of induced expression of the target gene (e.g., limited tetracycline-inducible promoter activity in the presence of a tetracycline). Thus, there remains a continued need for improved tetracycline-inducible expression systems with (i) reduced background expression of target genes (e.g., expression in the absence of a tetracycline); (ii) increased expression of target genes (e.g., in the presence of tetracycline); and/or (iii) increased fold induction of target gene expression (increased ratio of induced target gene expression relative to background expression).

The present disclosure provides, among other things, methods for controlling expression of a target gene in a mammalian cell and vectors comprising a minimal promoter and a TetO site array in which TetO sites are spaced more compactly and with more TetO sites than in pTRE3GV. Such technologies provide improved tetracycline-inducible gene expression systems with (i) reduced background expression of target genes (e.g., expression in the absence of a tetracycline); (ii) increased expression of target genes (e.g., in the presence of a tetracycline); and/or (iii) increased fold induction of target gene expression (increased ratio of induced target gene expression relative to background expression).

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the single forms “a”, “an”, and “the” are intended to include the plural forms as well, unless the context clearly indicates otherwise.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used here, the term “about,” when used to modify a numerical value, indicates that deviations of up to 10% above and below the numerical value, including the numerical value, remain within the intended meaning of the recited value. For example, “about 10” should be understood as both “10” and “9-11”.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “engineered” refers to the aspect of having been manipulated by the hand of man. For example, a nucleic acid is considered to be “engineered” when two or more sequences that are not linked together in that order in nature are manipulated by the hand of man to be directly linked to one another in the engineered nucleic acid and/or when a particular nucleotide residue in a nucleic acid is non-naturally occurring and/or is caused through action of the hand of man to be linked with an entity or moiety with which it is not linked in nature (e.g., a nucleic acid encoding a fusion polypeptide described herein). Comparably, a polypeptide (e.g., a transcriptional activator) can be considered to be “engineered” (e.g., an “engineered transcriptional activator”) if it has been subjected to a manipulation, so that its genetic, epigenetic, and/or phenotypic identity is altered relative to an appropriate reference polypeptide such as an otherwise identical polypeptide that has not been so manipulated. In some embodiments, the manipulation is or comprises a genetic manipulation, so that its genetic information is altered (e.g., new genetic material not previously present has been introduced, for example by transformation, transfection, transduction, or other mechanism, or previously present genetic material is altered or removed, for example by substitution or deletion mutation) or by chemical or physical manipulation (e.g., exposure to a specific biomolecule, such as lipids in growth medium). As is common practice and is understood by those in the art, progeny of, for example, an engineered polynucleotide or cell are typically still referred to as “engineered” even though the actual manipulation was performed on a prior entity.

As used herein, the term “expression” of a nucleic acid sequence refers to the generation of any gene product from the nucleic acid sequence. A gene product can be a transcript. A gene product can be a polypeptide. Expression of a nucleic acid sequence can involve one or more of the following: (1) production of an RNA template from a DNA sequence (e.g., by transcription); (2) processing of an RNA transcript (e.g., by splicing, editing, etc.); (3) translation of an RNA into a polypeptide; and/or (4) post-translational modification of a polypeptide.

As used herein, the terms “improved”, “increased”, or “reduced”, or grammatically comparable comparative terms, indicate values that are relative to a comparable reference measurement. For example, in some embodiments, an assessed value achieved with an agent of interest (e.g., an inducible gene expression system of the present disclosure) may be “improved” relative to that obtained with a comparable reference agent (e.g., an inducible gene expression system known in the art, e.g., comprising pTRE3GV). Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject or system (e.g., in a comparable subject or system that differs from the subject or system of interest in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the term “operably linked”, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

The terms “polypeptide”, “peptide”, and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full-length proteins or fragments thereof, wherein the amino acid residues are linked by covalent peptide bonds.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

As used herein, a “reference” entity, system, amount, set of conditions, etc., is one against which a test entity, system, amount, set of conditions, etc. is compared as described herein. For example, in some embodiments, a “reference” inducible gene expression system is a control inducible gene expression system, e.g., a gene expression system comprising pTRE3GV. In some embodiments, a reference or control is tested and/or determined simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “sequence identity” refers to the subunit sequence identity between two polymeric molecules particularly between two amino acid molecules, such as, between two polypeptide molecules, or two nucleic acid molecules. When two nucleotide sequences have the same nucleotides at the same positions, then they are identical at that position. For example, if a position in each of two nucleic acid molecules is occupied by an cytosine, then the two polypeptides are identical. The identity or extent to which two nucleotide sequences have the same nucleotide at the same positions in an alignment is often expressed as a percentage. The identity between two nucleotide sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “TetO site array” refers to a nucleic acid comprising a nucleotide sequence comprising two or more TetO sites.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to one another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

As used herein, the term “vector” has the same meaning as commonly understood by one of ordinary skill in the art, and refers to a nucleic acid molecule capable of transporting another nucleic acid molecule to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors used in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Methods of Controlling Expression of a Target Gene

Inducible gene expression systems enable tunable control of target gene expression (e.g., in mammalian cells). For example, tetracycline-inducible gene expression systems, such as DOX-inducible gene expression systems, comprise a transcriptional activator (e.g., an engineered transcriptional activator) and a tetracycline-inducible promoter to which the transcriptional activator binds to induce transcription of a target gene. In such systems, the transcriptional activator can bind to its cognate DNA operator site (e.g., tetracycline operator (TetO) site) within the tetracycline-inducible promoter when the transcriptional activator is bound to a tetracycline, whereas the transcriptional activator is understood not to bind to its cognate DNA operate site in the absence of (e.g., when unbound) to a tetracycline. A canonical tetracycline-inducible promoter comprises seven TetO sites spaced 17 base pairs apart and located upstream (5′ on the nucleic acid) of a minimal promoter. Thus, tetracycline-inducible expression systems comprise (i) a transcriptional activator (or a nucleic acid comprising a nucleotide sequence encoding a transcriptional activator); and (ii) a tetracycline-inducible promoter that controls expression of a target get in mammalian cells.

Tetracycline-inducible gene expression systems are particularly useful for activating expression of target genes stably integrated in the genome of mammalian cells. However, a continued challenge with such tetracycline-inducible gene expression systems is “leaky” background expression (e.g., tetracycline-inducible promoter activity in the absence of a tetracycline) and a limited upper bound of induced expression of the target gene (e.g., limited tetracycline-inducible promoter activity in the presence of a tetracycline). Thus, there remains a continued need for improved tetracycline-inducible expression systems with (i) reduced background expression of target genes (e.g., expression in the absence of a tetracycline); (ii) increased expression of target genes (e.g., in the presence of a tetracycline); and/or (iii) increased fold induction of target gene expression (increased ratio of induced target gene expression relative to background expression).

The present disclosure provides, among other things, improved methods for controlling expression of a target gene in a mammalian cell, comprising: (a) transiently transfecting or stably genomically integrating a mammalian cell with (1) a first nucleic acid comprising a nucleotide sequence comprising a tetracycline-inducible promoter that controls expression of the target gene; and (2) a second nucleic acid comprising a nucleotide sequence encoding a transcriptional activator that binds to TetO sites, thereby producing an engineered mammalian cells; and (b) contacting the engineering mammalian cell with a tetracycline to induce expression of the target gene, wherein the tetracycline-inducible promoter comprises a minimal promoter and a TetO site array in which TetO sites are spaced more compactly and with more TetO sites than in pTRE3GV. Methods for controlling expression of a target gene in a mammalian cell of the present disclosure can result in (i) reduced background expression of target genes (e.g., expression in the absence of tetracycline); (ii) increased expression of target genes (e.g., in the presence of tetracycline); and/or (iii) increased fold induction of target gene expression (increased ratio of induced target gene expression relative to background expression).

In some embodiments, a vector comprises the first and second nucleic acid (e.g., as described elsewhere herein). In some embodiments, a first vector comprises the first nucleic acid and a second vector comprises the second nucleic acid (e.g., as described elsewhere herein).

In some embodiments, a tetracycline for use in accordance with the technologies of the present disclosure comprises doxycycline. In some embodiments, technologies of the present disclosure comprising contacting an engineered mammalian cell with about 1 ng/ml to about 1 ug/mL, about 1 ng/mL to about 750 ng/mL, about 1 ng/mL to about 500 ng/ml, about 1 ng/ml to about 250 ng/mL, about 1 ng/mL to about 100 ng/ml, about 10 ng/ml to about 1 μg/mL, about 10 ng/mL to about 750 ng/ml, about 10 ng/mL to about 500 ng/ml, about 10 ng/mL to about 250 ng/ml, about 10 ng/mL to about 100 ng/mL of a tetracycline (e.g., doxycycline). In some embodiments, technologies of the present disclosure comprising contacting an engineered mammalian cell with about 1 ng/ml of a tetracycline (e.g., doxycycline). In some embodiments, technologies of the present disclosure comprising contacting an engineered mammalian cell with about 10 ng/ml of a tetracycline (e.g., doxycycline). In some embodiments, technologies of the present disclosure comprising contacting an engineered mammalian cell with about 100 ng/ml of a tetracycline (e.g., doxycycline). In some embodiments, technologies of the present disclosure comprising contacting an engineered mammalian cell with about 250 ng/mL of a tetracycline (e.g., doxycycline). In some embodiments, technologies of the present disclosure comprising contacting an engineered mammalian cell with about 500 ng/ml of a tetracycline (e.g., doxycycline). In some embodiments, technologies of the present disclosure comprising contacting an engineered mammalian cell with about 750 ng/ml of a tetracycline (e.g., doxycycline). In some embodiments, technologies of the present disclosure comprising contacting an engineered mammalian cell with about 1 μg/mL of a tetracycline (e.g., doxycycline).

In some embodiments, methods of controlling expression of a target gene in a mammalian cell are conducted under conditions sufficient to permit (i) uptake of the first and second nucleic acids by the mammalian cell; and (ii) translation of the transcriptional activator and target gene. Such conditions are well known in the art and the skilled artisan would recognize a variety of techniques and/or cell culture conditions that could be successfully utilized. Such techniques include, for example, and without limitation, microinjection, bolistic methods, laserfection/optical transfection, transduction (e.g., using viral vectors), and transfection. The choice of such techniques and/or cell culture conditions can depend on a plurality of factors, such as the type and origin of cells and the form of contacted nucleic acid. Selection of such techniques and/or cell culture conditions are well within the level of the skilled artisan.

### Target Genes

As will be apparent to those of ordinary skill in the art, target genes of the present disclosure can comprise (or encode) any gene (or protein) known in the art. Non-limiting examples of target genes of the present disclosure include genes that encode therapeutic polypeptides or polypeptides useful in biomedical research. Therapeutic polypeptides can include, for example and without limitation, enzymes (e.g., nucleases), monoclonal antibodies, transcription factors, antigens (e.g., for use in vaccines), fusion proteins, gene therapy vectors (e.g., adeno-associated virus (AAV)) and/or cytokines. Polypeptides useful in biomedical research can include, for example and without limitation, enzymes (e.g., horse-radish peroxidase), fluorescent proteins (e.g., blue fluorescent protein, green fluorescent protein, mCherry, dsRedExpress2, mNeonGreen), transcription factors, and antibodies.

### First Nucleic Acids of the Present Disclosure and Tetracycline-Inducible Promoters

A first nucleic acid of the present disclosure can comprise a nucleotide sequence comprising a tetracycline-inducible promoter that controls expression of a target gene in a mammalian cell. The tetracycline-inducible promoter can comprise a promoter (e.g., a minimal promoter) and a TetO site array in which TetO sites are spaced more compactly and with more TetO sites than in pTRE3GV (SEQ ID NO: 1). In some embodiments, the TetO site array is located upstream (5′ on the nucleic acid) of the promoter (e.g., minimal promoter).

The TetO site array of the first nucleic acid of the present disclosure can comprise 8 or more TetO sites. In some embodiments, the TetO site array comprises 8, 9, 10, 11, 12, 13, 14, or 15 TetO sites. In some embodiments, the TetO site array comprises 8 TetO sites. In some embodiments, the TetO site array comprises 9 TetO sites. In some embodiments, the TetO site array comprises 10 TetO sites. In some embodiments, the TetO site array comprises 11 TetO sites. In some embodiments, the TetO site array comprises 12 TetO sites. In some embodiments, the TetO site array comprises 13 TetO sites. In some embodiments, the TetO site array comprises 14 TetO sites. In some embodiments, the TetO site array comprises 15 TetO sites.

The TetO site array of the first nucleic acid of the present disclosure can comprise TetO sites which are spaced more compactly than the TetO sites in pTRE3GV (SEQ ID NO: 1). For example, each TetO site in a TetO site array of the present disclosure can be less than 17 base pairs apart. In some embodiments, each TetO site in a TetO array of the present disclose are less than 6 base pairs apart. In some embodiments, each TetO site in a TetO site array of the present disclosure is 2-17 base pairs apart, 2-15 base pairs apart, 2-12 base pairs apart, 2-10 base pairs apart, 2-8 base pairs apart, 2-6 base pairs apart, 4-17 base pairs apart, 6-17 base pairs apart, 8-17 base pairs apart, 10-17 base pairs apart, or 12-17 base pairs apart. In some embodiments, each TetO site in a TetO site array of the present disclosure is 17 base pairs apart. In some embodiments, each TetO site in a TetO site array of the present disclosure is 16 base pairs apart. In some embodiments, each TetO site in a TetO site array of the present disclosure is 15 base pairs apart. In some embodiments, each TetO site in a TetO site array of the present disclosure is 14 base pairs apart. In some embodiments, each TetO site in a TetO site array of the present disclosure is 13 base pairs apart. In some embodiments, each TetO site in a TetO site array of the present disclosure is 12 base pairs apart. In some embodiments, each TetO site in a TetO site array of the present disclosure is 11 base pairs apart. In some embodiments, each TetO site in a TetO site array of the present disclosure is 10 base pairs apart. In some embodiments, each TetO site in a TetO site array of the present disclosure is 9 base pairs apart. In some embodiments, each TetO site in a TetO site array of the present disclosure is 8 base pairs apart. In some embodiments, each TetO site in a TetO site array of the present disclosure is 7 base pairs apart. In some embodiments, each TetO site in a TetO site array of the present disclosure is 6 base pairs apart. In some embodiments, each TetO site in a TetO site array of the present disclosure is 5 base pairs apart. In some embodiments, each TetO site in a TetO site array of the present disclosure is 4 base pairs apart. In some embodiments, each TetO site in a TetO site array of the present disclosure is 3 base pairs apart. In some embodiments, each TetO site in a TetO site array of the present disclosure is 2 base pairs apart.

A TetO site array of the present disclosure can comprise, for example, the sequence of SEQ ID NO: 7 or a variant thereof. Tccctatcagtgatagagaaacgcctccctatcagtgatagagaaaagcttccctatcagtgatagagaagaagaggtgtattccctat cagtgatagagaaacgcctccctatcagtgatagagaaaagcttccctatcagtgatagagaagatgtgaggtattccctatcagtgata gagaaacgcctccctatcagtgatagagaaaagcttccctatcagtgatagagaataggcctagtattccctatcagtgatagagaaac gcctccctatcagtgatagagaaaagcttccctatcagtgatagagaaACGCTC (SEQ ID NO: 7).

The tetracycline-inducible promoter of the present disclosure can comprise a promoter, such as a minimal promoter. The choice of promoter can be influenced by, for example, the choice of expression system (e.g., in vitro, in vivo, the particular mammalian cell-type) and/or the desired strength of target gene expression. In some embodiments, tetracycline-inducible promoter of the present disclosure comprises a minimal promoter. Minimal promoters, or “core promoters”, are short nucleic acid sequences that allow for the formation of a transcription initiation complex right at the transcription start site. In some embodiments, the minimal promoter is a YB_TATA minimal promoter or a variant thereof. The YB_TATA promoter can comprise, for example, the sequence of SEQ ID NO: 8 (TCTAGAGGGTATATAATGGGGGCCA) or a variant thereof. In some embodiments, the minimal promoter is a mini CMV minimal promoter or a variant thereof. In some embodiments, the minimal promoter is a miniTK minimal promoter or a variant thereof. In some embodiments, the minimal promoter is a synthetic minimal promoter. See, e.g., Greenshpan Y et al., Int J Mol Sci. 2022 Jul. 4; 23(13):7431, Ede C. et al., ACS Synth Biol. 2016 May 20; 5(5):395-404, Hansen et al., PNAS. 2014 Oct. 20; 111 (44) 15705-15710.

In some embodiments, a first nucleic acid of the present disclosure further comprises a GC box. A GC box is a transcriptional regulatory element comprising the sequence GGGCGG. GC boxes are often found within 100 bases upstream of the transcriptional start site, and although they may appear as a single instance, they are commonly repeated 20 to 50 times.

In some embodiments, a first nucleic acid of the present disclosure further comprises a GC box upstream from the promoter (e.g., minimal promoter) of the tetracycline-inducible promoter. In some embodiments, a first nucleic acid of the present disclosure further comprises a GC box about 1 to about 100 bases upstream, about 1 to about 80 bases upstream, about 1 to about 60 bases upstream, about 1 to about 50 bases upstream, about 1 to about 40 bases upstream, about 1 to about 25 bases upstream, about 1 to about 10 bases upstream, about 10 to about 100 bases upstream, about 10 to about 80 bases upstream, about 10 to about 60 bases upstream, about 10 to about 50 bases upstream, about 10 to about 40 bases upstream, or about 10 to about 25 bases upstream from the promoter (e.g., minimal promoter) of the tetracycline-inducible promoter. In some embodiments, a first nucleic acid of the present disclosure further comprises a GC box about 5 base pairs upstream from the promoter of the tetracycline-inducible promoter. In some embodiments, a first nucleic acid of the present disclosure further comprises a GC box about 10 base pairs upstream from the promoter of the tetracycline-inducible promoter. In some embodiments, a first nucleic acid of the present disclosure further comprises a GC box about 20 base pairs upstream from the promoter of the tetracycline-inducible promoter. In some embodiments, a first nucleic acid of the present disclosure further comprises a GC box about 30 base pairs upstream from the promoter of the tetracycline-inducible promoter. In some embodiments, a first nucleic acid of the present disclosure further comprises a GC box about 40 base pairs upstream from the promoter of the tetracycline-inducible promoter. In some embodiments, a first nucleic acid of the present disclosure further comprises a GC box about 50 base pairs upstream from the promoter of the tetracycline-inducible promoter. In some embodiments, a first nucleic acid of the present disclosure further comprises a GC box about 75 base pairs upstream from the promoter of the tetracycline-inducible promoter. In some embodiments, a first nucleic acid of the present disclosure further comprises a GC box about 100 base pairs upstream from the promoter of the tetracycline-inducible promoter.

In some embodiments, the first nucleic acid of the present disclosure comprises 1-50 GC boxes. In some embodiments, the first nucleic acid of the present disclosure comprises 1-50, 5-50, 10-50, 15-50, 20-50, 25-50, 30-50, 1-40, 5-40, 10-40, 15-40, 20-40, 25-40, 30-40, 1-25, 5-25, 10-25, or 15-20 GC boxes. In some embodiments, the first nucleic acid of the present disclosure comprises 1 GC box. In some embodiments, the first nucleic acid of the present disclosure comprises 2 GC boxes. In some embodiments, the first nucleic acid of the present disclosure comprises 5 GC boxes. In some embodiments, the first nucleic acid of the present disclosure comprises 10 GC boxes. In some embodiments, the first nucleic acid of the present disclosure comprises 15 GC boxes. In some embodiments, the first nucleic acid of the present disclosure comprises 20 GC boxes. In some embodiments, the first nucleic acid of the present disclosure comprises 30 GC boxes. In some embodiments, the first nucleic acid of the present disclosure comprises 40 GC boxes. In some embodiments, the first nucleic acid of the present disclosure comprises 50 GC boxes.

In some embodiments, the first nucleic acid of the present disclosure comprises a nucleotide sequence having about 70%, about 75%, about 80%, about 85%, about 90%, about 95%, about 96%, about 97%, about 98%, or about 99% identity to SEQ ID NO: 2. In some embodiments, the first nucleic acid of the present disclosure comprises the nucleotide sequence of SEQ ID NO: 2.

In some embodiments, the first nucleic acid of the present disclosure comprises a nucleotide sequence having about 70%, about 75%, about 80%, about 85%, about 90%, about 95%, about 96%, about 97%, about 98%, or about 99% identity to SEQ ID NO: 3. In some embodiments, the first nucleic acid of the present disclosure comprises the nucleotide sequence of SEQ ID NO: 3.

In some embodiments, the first nucleic acid of the present disclosure comprises a nucleotide sequence having about 70%, about 75%, about 80%, about 85%, about 90%, about 95%, about 96%, about 97%, about 98%, or about 99% identity to SEQ ID NO: 4. In some embodiments, the first nucleic acid of the present disclosure comprises the nucleotide sequence of SEQ ID NO: 4.

In some embodiments, the first nucleic acid of the present disclosure comprises a nucleotide sequence having about 70%, about 75%, about 80%, about 85%, about 90%, about 95%, about 96%, about 97%, about 98%, or about 99% identity to SEQ ID NO: 5. In some embodiments, the first nucleic acid of the present disclosure comprises the nucleotide sequence of SEQ ID NO: 5.

In some embodiments, a tetracycline-inducible promoter of the present disclosure exhibits reduced promoter activity in the absence of a tetracycline (e.g., compared to an appropriate reference, e.g., the tetracycline-inducible promoter in the presence of a tetracycline, pTRE3GV in the absence of a tetracycline). For example, a tetracycline-inducible promoter of the present disclosure can exhibit a reduced promoter activity of about 99%, 98%, 97%, 95%, 90%, 85%, 80%, 75%, 70%, 60%, 50%, 40%, 30%, 20% or 10% as compared to an appropriate reference.

In some embodiments, a tetracycline-inducible promoter of the present disclosure provides a higher expression level of a target gene than an appropriate reference, such as pTRE3GV. For example, a tetracycline-inducible promoter of the present disclosure can provide a high expression level of a target gene by about 2-fold, about 3-fold, about 4-fold, about 5-fold, about 6-fold, about 7-fold, about 8-fold, about 10-fold, about 15-fold, about 20-fold, about 25-fold, or about 50-fold compared to an appropriate reference (e.g., gene expression resulting from a pTRE3GV comprising gene expression system).

In some embodiments, a tetracycline-inducible promoter of the present disclosure provides a fold induction in expression level of the target gene upon treatment with a tetracycline over background expression level that is higher compared to a reference (e.g., fold induction resulting from a pTRE3GV comprising gene expression system). Such a fold-induction can be, for example, about 2-fold, about 3-fold, about 4-fold, about 5-fold, about 6-fold, about 7-fold, about 8-fold, about 10-fold, about 15-fold, about 20-fold, about 25-fold, about 50-fold, or higher than that provided by the appropriate reference. In some embodiments, the fold induction in expression level of the target gene upon treatment with tetracycline over background expression level that is more than 2-fold higher than that provided by pTRE3GV. In some embodiments, the fold induction in expression level of the target gene upon treatment with tetracycline over background expression level that is more than 3-fold higher than that provided by pTRE3GV. In some embodiments, the fold induction in expression level of the target gene upon treatment with tetracycline over background expression level that is more than 4-fold higher than that provided by pTRE3GV. In some embodiments, the fold induction in expression level of the target gene upon treatment with tetracycline over background expression level that is more than 5-fold higher than that provided by pTRE3GV. In some embodiments, the fold induction in expression level of the target gene upon treatment with tetracycline over background expression level that is more than 6-fold higher than that provided by pTRE3GV. In some embodiments, the fold induction in expression level of the target gene upon treatment with tetracycline over background expression level that is more than 7-fold higher than that provided by pTRE3GV. In some embodiments, the fold induction in expression level of the target gene upon treatment with tetracycline over background expression level that is more than 8-fold higher than that provided by pTRE3GV. In some embodiments, the fold induction in expression level of the target gene upon treatment with tetracycline over background expression level that is more than 10-fold higher than that provided by pTRE3GV. In some embodiments, the fold induction in expression level of the target gene upon treatment with tetracycline over background expression level that is more than 15-fold higher than that provided by pTRE3GV. In some embodiments, the fold induction in expression level of the target gene upon treatment with tetracycline over background expression level that is more than 20-fold higher than that provided by pTRE3GV. In some embodiments, the fold induction in expression level of the target gene upon treatment with tetracycline over background expression level that is more than 25-fold higher than that provided by pTRE3GV. In some embodiments, the fold induction in expression level of the target gene upon treatment with tetracycline over background expression level that is more than 50-fold higher than that provided by pTRE3GV.

Methods to evaluate promoter activity and expression levels (e.g., target gene expression level, background expression level) are well known in the art and their selection and use in accordance with technologies of the present disclosure are well within the level of one of ordinary skill in the art.

### Second Nucleic Acids of the Present Disclosure and Transcriptional Activators

A second nucleic acid of the present disclosure can comprise a nucleotide sequence encoding a transcriptional activator that can bind to a TetO site. As used herein, the term “transcriptional activator” (also referred to as “transactivators”) refers to a molecule (e.g., a polypeptide) capable of activating expression of a target gene from an operably linked promoter (e.g., a tetracycline-inducible promoter, e.g., a doxycycline-inducible promoter). In effect, the transcriptional activator “activates” the expression of the target gene from its tetracycline-inducible promoter.

Thus, according to one aspect of technologies of the present disclosure, a tetracycline (e.g., a doxycycline) is provided that binds to the transcriptional activator causing a conformational change in the transcriptional activator, thereby rendering it able to bind to a TetO site (e.g., within a TetO site array) and induce expression of the target gene.

Transcriptional activators and their cognate binding sites are known to those of ordinary skill in the art and include, for example, variants of the tetracycline repressor protein (TetR) (e.g., the bacterial TetR), such as the reverse Tet repressor (rTetR) and variants thereof (e.g., fused to a transcriptional activation domain, such as VP16), reverse tetracycline transactivator (rtTA) protein and variants thereof, rtTA2s-M2 (abbreviated as M2; also referred to as “Tet-On advanced”, Clontech), Tet-On 3G transactivator protein (Takara Bio). See, e.g., Das A T, et al. Curr Gene Ther. 2016; 16(3):156-67, WO 2020/069339, WO2000075347, incorporated herein by reference.

### Engineered Mammalian Cells

Engineered mammalian cells produced in accordance with technologies of the present disclosure or a plurality thereof are mammalian cells that have been transiently transfected or stably genomically integrated with a first and second nucleic acid described herein. Engineered mammalian cells can be or comprise any mammalian cell type, including, for example, HEK293 cells, HEK293FT cells, Chinese Hamster Ovary (CHO) cells, HeLa cells, HepG2 cells, COS cells, and Vero cells.

### Vectors

A first nucleic acid comprising a nucleotide sequence comprising a tetracycline-inducible promoter that controls expression of a target gene and a second nucleic acid comprising a nucleotide sequence encoding a transcriptional activator (e.g., that binds to TetO sites) of the present disclosure are recombinant polynucleotides (e.g., DNA). Such recombinant polynucleotides may be prepared by a variety of methods available and known in the art. For example, nucleic acids of the present disclosure may be excised from DNA using restriction enzymes, may be amplified from plasmids or genomic polynucleotide sequences using, for example, polymerase chain reaction, or may be synthesized (e.g., using chemical synthesis techniques or in vitro transcription). A combination of known methods may also be utilized to prepare a nucleic acid of the present disclosure.

A first nucleic acid comprising a nucleotide sequence comprising a tetracycline-inducible promoter that controls expression of a target gene and a second nucleic acid comprising a nucleotide sequence encoding a transcriptional activator of the present disclosure can be cloned into a single vector or separate vectors (e.g., a vector capable of expressing a target gene, the transcriptional activator). A variety of suitable cloning methods are known in the art and their use is well within the level of one of ordinary skill in the art.

Vectors for use in accordance with the present disclosure can be non-viral or viral vectors. The choice of expression vector can be influenced by the choice of expression system (e.g., in vitro, in vivo, cell-type). Such selection is well within the level of skill of one of ordinary skill in the art. In general, vectors can include transcriptional promoters and optionally, for example, enhancers, translation signals, and transcriptional and translation termination signals. Vectors that are used for stable transformation typically have a selectable marker which allows selection and maintenance of the transformed cells. In some embodiments, an origin of replication can be used to amplify the copy number of the vector in the cells.

In some embodiments, a vector (e.g., a single vector) comprises the first and second nucleic acids of the present disclosure. In some embodiments, the vector is a non-viral vector (e.g., a plasmid). In some embodiments, the vector is a viral vector. In some such embodiments, the viral vector is a lentiviral vector.

In some embodiments, a first vector comprises the first nucleic acid and the second vector comprises the second nucleic acid. In some embodiments, the first vector and the second vector are both a non-viral vector (e.g., plasmids). In some embodiments, the first vector and the second vector are both a viral vector. In some embodiments, the first vector is a non-viral vector (e.g., a plasmid) and the second vector is a viral vector. In some embodiments, the first vector is a viral vector and the second vector is a non-viral vector (e.g., a plasmid). In some such embodiments, a viral vector is a lentiviral vector.

In some embodiments, wherein a second vector comprises second the second nucleic acid, the second nucleic acid further comprises a nucleotide sequence comprising a constitutive promoter. In some such embodiments, the constitutive promoter comprises a tissue-specific or cell state-specific promoter.

Vectors can further comprise additional nucleic acids operably linked to a nucleic acid molecule (e.g., encoding a transcriptional activator as described herein, encoding a target gene as described herein), such as, for example, an epitope tag (e.g., for visualization, such as a fluorescent tag) and/or a tag for purification (e.g., a His tag).

Expression of transcriptional activators and/or target genes of the present disclosure can be controlled by any suitable promoter (e.g., minimal promoter) and/or enhancer known in the art. Suitable promoters for mammalian cells are well known in the art. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bemoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 75:1441-1445 (1981)), the regulatory sequences of the metallothionein gene (Brinster et al., Nature 296:39-42 (1982)); prokaryotic expression vectors such as the b-lactamase promoter (Jay et al., Proc. Natl. Acad. Sci. USA 75:5543 (1981)) or the tac promoter (DeBoer et al., Proc. Natl. Acad. Sci. USA 50:21-25 (1983)); see also “Useful Proteins from Recombinant Bacteria”: in Scientific American 242:79-94 (1980)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515:115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7:1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al, Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al, Science 254:1372-1378 (1986)). In some embodiments, a vector comprises the cytomegalovirus (CMV) promoter, such as a minimal CMV promoter or a variant thereof. In some embodiments, a vector comprises a minimal promoter. In some embodiments, the minimal promoter is a YB_TATA minimal promoter or a variant thereof. In some embodiments, the minimal promoter is a variant of a minimal CMV promoter. In some embodiments, the minimal promoter is a mini CMV minimal promoter or a variant thereof. In some embodiments, the minimal promoter is a miniTK minimal promoter or a variant thereof. In some embodiments, a vector comprises a synthetic minimal promoter. In some embodiments, the minimal promoter is a variant of a minimal CMV promoter or a synthetic minimal promoter.

In addition to the promoter, an expression vector typically contains a transcription unit or expression cassette that contains all the additional elements required for the expression of a transcriptional activator and/or target gene. A typical expression cassette contains a promoter operably linked to the nucleic acid sequence encoding the transcriptional activator and/or target gene and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

In one aspect, the present disclosure provides a vector comprising a minimal promoter and a TetO site array in which TetO sites are spaced more compactly and with more TetO sites than in pTRE3GV (SEQ ID NO: 1).

The TetO site array of a vector of the present disclosure can comprise 8 or more TetO sites. In some embodiments, the TetO site array comprises 8, 9, 10, 11, 12, 13, 14, or 15 TetO sites. In some embodiments, the TetO site array comprises 8 TetO sites. In some embodiments, the TetO site array comprises 9 TetO sites. In some embodiments, the TetO site array comprises 10 TetO sites. In some embodiments, the TetO site array comprises 11 TetO sites. In some embodiments, the TetO site array comprises 12 TetO sites. In some embodiments, the TetO site array comprises 13 TetO sites. In some embodiments, the TetO site array comprises 14 TetO sites. In some embodiments, the TetO site array comprises 15 TetO sites.

The TetO site array of a vector of the present disclosure can comprise TetO sites which are spaced more compactly than the TetO sites in pTRE3GV (SEQ ID NO: 1). For example, each TetO site in a TetO site array of the present disclosure can be less than 17 base pairs apart. In some embodiments, each TetO site in a TetO site array of the present disclosure is 2-17 base pairs apart, 2-15 base pairs apart, 2-12 base pairs apart, 2-10 base pairs apart, 2-8 base pairs apart, 2-6 base pairs apart, 4-17 base pairs apart, 6-17 base pairs apart, 8-17 base pairs apart, 10-17 base pairs apart, or 12-17 base pairs apart. In some embodiments, each TetO site in a TetO site array of the present disclosure is 17 base pairs apart. In some embodiments, each TetO site in a TetO site array of the present disclosure is 15 base pairs apart. In some embodiments, each TetO site in a TetO site array of the present disclosure is 12 base pairs apart. In some embodiments, each TetO site in a TetO site array of the present disclosure is 10 base pairs apart. In some embodiments, each TetO site in a TetO site array of the present disclosure is 8 base pairs apart. In some embodiments, each TetO site in a TetO site array of the present disclosure is 6 base pairs apart. In some embodiments, each TetO site in a TetO site array of the present disclosure is 4 base pairs apart. In some embodiments, each TetO site in a TetO site array of the present disclosure is 2 base pairs apart.

A vector (e.g., a first vector) of the present disclosure comprises a minimal promoter. In some embodiments, the minimal promoter is a YB_TATA minimal promoter or a variant thereof. In some embodiments, the minimal promoter is a mini CMV minimal promoter of a variant thereof. In some embodiments, the minimal promoter is a miniTK minimal promoter or a variant thereof. In some embodiments, the minimal promoter is a synthetic minimal promoter. In some embodiments, the minimal promoter is a minimal CMV promoter or a variant thereof. In some embodiments, the minimal promoter is a variant of a minimal CMV promoter or a synthetic minimal promoter.

In some embodiments, a vector of the present disclosure further comprises a GC box. A GC box is a transcriptional regulatory element comprising the sequence GGGCGG. GC boxes are often found within 100 bases upstream of the transcriptional start site, and although they may appear as a single instance, they are commonly repeated 20 to 50 times.

In some embodiments, a vector of the present disclosure further comprises a GC box upstream from the promoter (e.g., minimal promoter) of the tetracycline-inducible promoter. In some embodiments, a vector of the present disclosure further comprises a GC box about 1 to about 100 bases upstream, about 1 to about 80 bases upstream, about 1 to about 60 bases upstream, about 1 to about 50 bases upstream, about 1 to about 40 bases upstream, about 1 to about 25 bases upstream, about 1 to about 10 bases upstream, about 10 to about 100 bases upstream, about 10 to about 80 bases upstream, about 10 to about 60 bases upstream, about 10 to about 50 bases upstream, about 10 to about 40 bases upstream, or about 10 to about 25 bases upstream from the promoter (e.g., minimal promoter). In some embodiments, a vector of the present disclosure further comprises a GC box about 5 base pairs upstream from the promoter. In some embodiments, a vector of the present disclosure further comprises a GC box about 10 base pairs upstream from the promoter. In some embodiments, a vector of the present disclosure further comprises a GC box about 20 base pairs upstream from the promoter. In some embodiments, a vector of the present disclosure further comprises a GC box about 30 base pairs upstream from the promoter. In some embodiments, a vector of the present disclosure further comprises a GC box about 40 base pairs upstream from the promoter. In some embodiments, a vector of the present disclosure further comprises a GC box about 50 base pairs upstream from the promoter. In some embodiments, a vector of the present disclosure further comprises a GC box about 75 base pairs upstream from the promoter. In some embodiments, a vector of the present disclosure further comprises a GC box about 100 base pairs upstream from the promoter.

In some embodiments, the vector of the present disclosure comprises 1-50 GC boxes. In some embodiments, the vector of the present disclosure comprises 1-50, 5-50, 10-50, 15-50, 20-50, 25-50, 30-50, 1-40, 5-40, 10-40, 15-40, 20-40, 25-40, 30-40, 1-25, 5-25, 10-25, or 15-20 GC boxes. In some embodiments, the vector of the present disclosure comprises 1 GC box. In some embodiments, the first nucleic acid of the present disclosure comprises 2 GC boxes. In some embodiments, the first nucleic acid of the present disclosure comprises 5 GC boxes. In some embodiments, the first nucleic acid of the present disclosure comprises 10 GC boxes. In some embodiments, the first nucleic acid of the present disclosure comprises 15 GC boxes. In some embodiments, the first nucleic acid of the present disclosure comprises 20 GC boxes. In some embodiments, the first nucleic acid of the present disclosure comprises 30 GC boxes. In some embodiments, the first nucleic acid of the present disclosure comprises 40 GC boxes. In some embodiments, the first nucleic acid of the present disclosure comprises 50 GC boxes.

In some embodiments, the vector of the present disclosure comprises a nucleotide sequence having about 70%, about 75%, about 80%, about 85%, about 90%, about 95%, about 96%, about 97%, about 98%, or about 99% identity to SEQ ID NO: 2. In some embodiments, the vector of the present disclosure comprises the nucleotide sequence of SEQ ID NO: 2.

In some embodiments, the vector of the present disclosure comprises a nucleotide sequence having about 70%, about 75%, about 80%, about 85%, about 90%, about 95%, about 96%, about 97%, about 98%, or about 99% identity to SEQ ID NO: 3. In some embodiments, the vector of the present disclosure comprises the nucleotide sequence of SEQ ID NO: 3.

In some embodiments, the vector of the present disclosure comprises a nucleotide sequence having about 70%, about 75%, about 80%, about 85%, about 90%, about 95%, about 96%, about 97%, about 98%, or about 99% identity to SEQ ID NO: 4. In some embodiments, the vector of the present disclosure comprises the nucleotide sequence of SEQ ID NO: 4.

In some embodiments, the vector of the present disclosure comprises a nucleotide sequence having about 70%, about 75%, about 80%, about 85%, about 90%, about 95%, about 96%, about 97%, about 98%, or about 99% identity to SEQ ID NO: 5. In some embodiments, the vector of the present disclosure comprises the nucleotide sequence of SEQ ID NO: 5.

In some embodiments, a vector of the present disclosure exhibits reduced promoter activity in the absence of a tetracycline (e.g., compared to an appropriate reference, e.g., the tetracycline-inducible promoter in the presence of a tetracycline, pTRE3GV in the absence of a tetracycline). For example, a vector of the present disclosure can exhibit a reduced promoter activity of about 99%, 98%, 97%, 95%, 90%, 85%, 80%, 75%, 70%, 60%, 50%, 40%, 30%, 20% or 10% as compared to an appropriate reference.

In some embodiments, a vector of the present disclosure provides a higher expression level of a target gene than an appropriate reference, such as pTRE3GV. For example, a vector of the present disclosure can provide a high expression level of a target gene by about 2-fold, about 3-fold, about 4-fold, about 5-fold, about 6-fold, about 7-fold, about 8-fold, about 10-fold, about 15-fold, about 20-fold, about 25-fold, or about 50-fold compared to an appropriate reference (e.g., gene expression resulting from a pTRE3GV comprising gene expression system).

In some embodiments, a vector of the present disclosure provides a fold induction in expression level of the target gene upon treatment with a tetracycline over background expression level that is higher compared to a reference (e.g., fold induction resulting from a pTRE3GV comprising gene expression system). Such a fold-induction can be, for example, about 2-fold, about 3-fold, about 4-fold, about 5-fold, about 6-fold, about 7-fold, about 8-fold, about 10-fold, about 15-fold, about 20-fold, about 25-fold, about 50-fold, or higher than that provided by the appropriate reference. In some embodiments, the fold induction in expression level of the target gene upon treatment with tetracycline over background expression level that is more than 2-fold higher than that provided by pTRE3GV. In some embodiments, the fold induction in expression level of the target gene upon treatment with tetracycline over background expression level that is more than 3-fold higher than that provided by pTRE3GV. In some embodiments, the fold induction in expression level of the target gene upon treatment with tetracycline over background expression level that is more than 4-fold higher than that provided by pTRE3GV. In some embodiments, the fold induction in expression level of the target gene upon treatment with tetracycline over background expression level that is more than 5-fold higher than that provided by pTRE3GV. In some embodiments, the fold induction in expression level of the target gene upon treatment with tetracycline over background expression level that is more than 6-fold higher than that provided by pTRE3GV. In some embodiments, the fold induction in expression level of the target gene upon treatment with tetracycline over background expression level that is more than 8-fold higher than that provided by pTRE3GV. In some embodiments, the fold induction in expression level of the target gene upon treatment with tetracycline over background expression level that is more than 10-fold higher than that provided by pTRE3GV. In some embodiments, the fold induction in expression level of the target gene upon treatment with tetracycline over background expression level that is more than 15-fold higher than that provided by pTRE3GV. In some embodiments, the fold induction in expression level of the target gene upon treatment with tetracycline over background expression level that is more than 20-fold higher than that provided by pTRE3GV. In some embodiments, the fold induction in expression level of the target gene upon treatment with tetracycline over background expression level that is more than 25-fold higher than that provided by pTRE3GV. In some embodiments, the fold induction in expression level of the target gene upon treatment with tetracycline over background expression level that is more than 50-fold higher than that provided by pTRE3GV.

Methods to evaluate promoter activity and expression levels (e.g., target gene expression level, background expression level) from a vector are well known in the art and their selection and use in accordance with technologies of the present disclosure are well within the level of one of ordinary skill in the art.

### Compositions and Kits

Among other things, the present disclosure also provides compositions comprising one or more of: (i) a first nucleic acid as described herein; (ii) a second nucleic acid as described herein; (iii) an engineered mammalian cell as described herein; (iv) a tetracycline; and (v) a vector as described herein.

In one aspect, the present disclosure provides kits comprising one or more containers comprising one or more of: (i) a first nucleic acid comprising a nucleotide sequence comprising a tetracycline-inducible promoter that controls expression of the target gene as described herein; (ii) a second nucleic acid comprising a nucleotide sequence encoding a transcriptional activator that binds to TetO sites as described herein; (iii) an engineered mammalian cell as described herein; (iv) a tetracycline; (v) a vector as described herein; (vi) a composition as described herein; and (vii) optionally, instructions for use or a link (e.g., QR code) to instructions for use.

A container may include, for example and without limitation, a vial, well, test tube, flask, bottle, syringe, infusion bag, or other container means. Where an additional component is included in the kit, the kit can contain additional containers into which this component may be placed. Containers and/or kits can comprise labeling with instructions for use and/or warnings.

### EXAMPLES

### Example 1: Evaluation of Doxycycline (DOX)-Inducible x12TetO Promoters Compared to pTRE3GV

The present example demonstrates, among other things, increased expression of a target gene and reduced background signal using DOX-inducible x12TetO promoters compared to pTRE3GV.

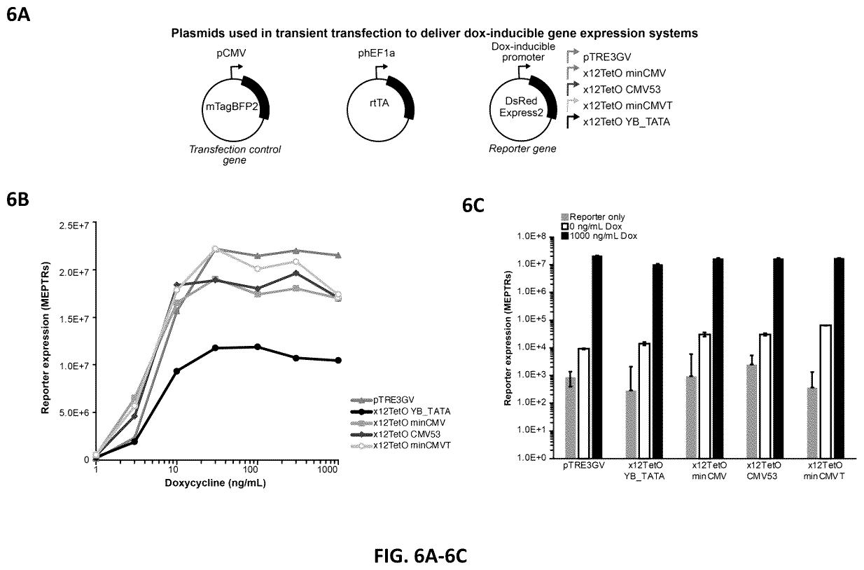

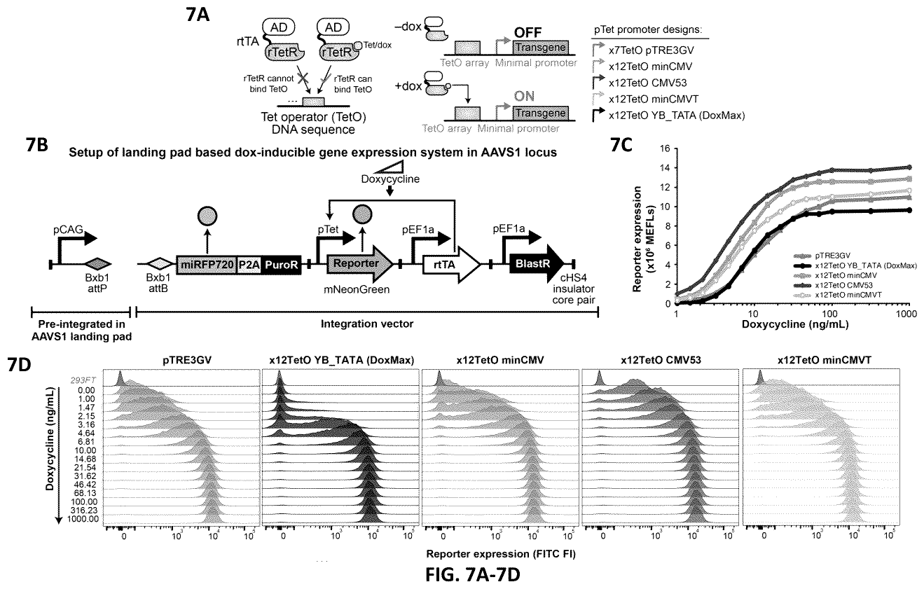

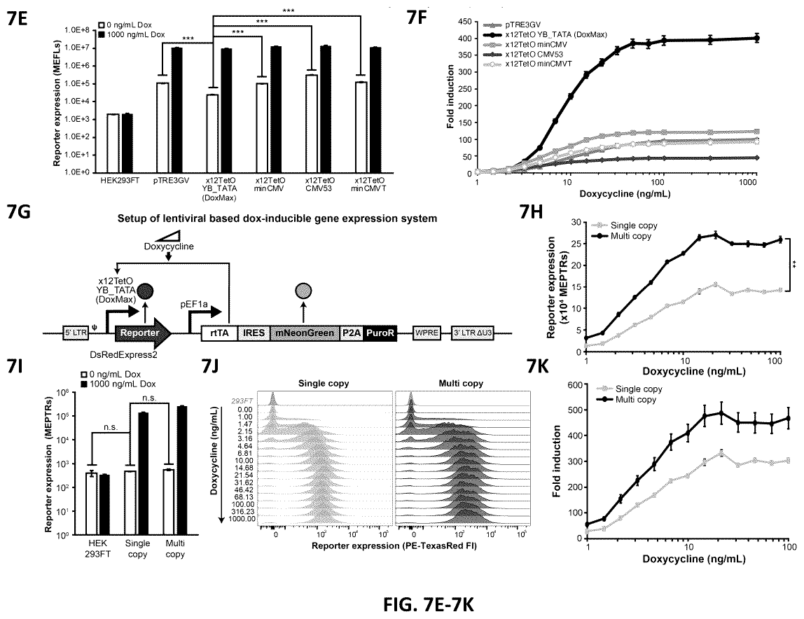

FIG. 7A provides a schematic of the Tet ON gene expression system, which employs the reverse tetracycline transactivator (rtTA), a fusion protein containing a transcriptional activation domain (AD) and a mutated form of the bacterial tetracycline repressor protein (rTetR). In the absence of tetracycline or analogs such as doxycycline (dox), rtTA cannot bind to its cognate DNA operator sequence. In the presence of dox, rtTA undergoes a conformational change that enables binding to its DNA operator sequence, subsequent recruitment of transcriptional machinery via its AD to the minimal promoter and resulting promoter activation and transgene expression. One challenge of the original Tet ON gene expression system is the leaky background gene expression in the absence of dox. To enhance this system, we paired a reported TetO site array with more TetO sites spaced more compactly with a panel of minimal promoters with different levels of basal activity.

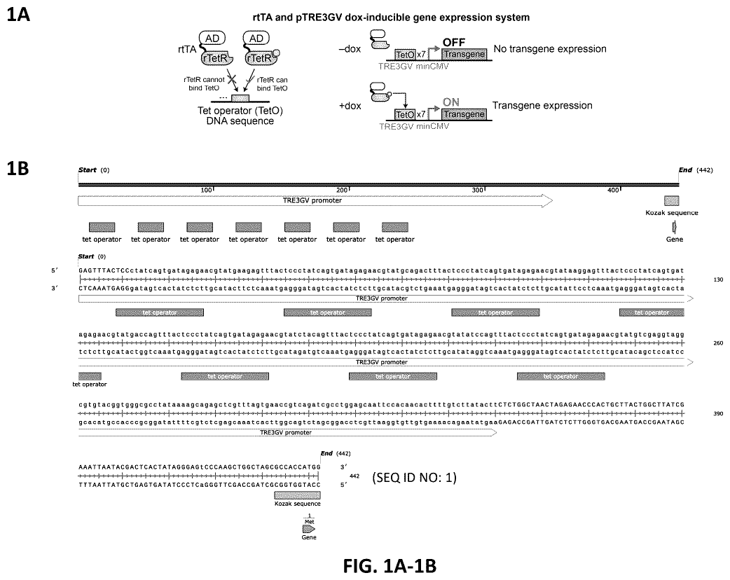

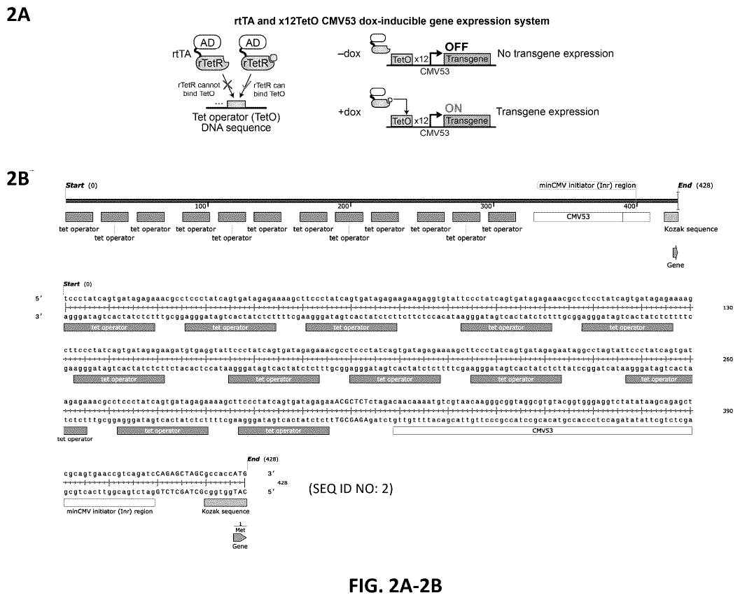

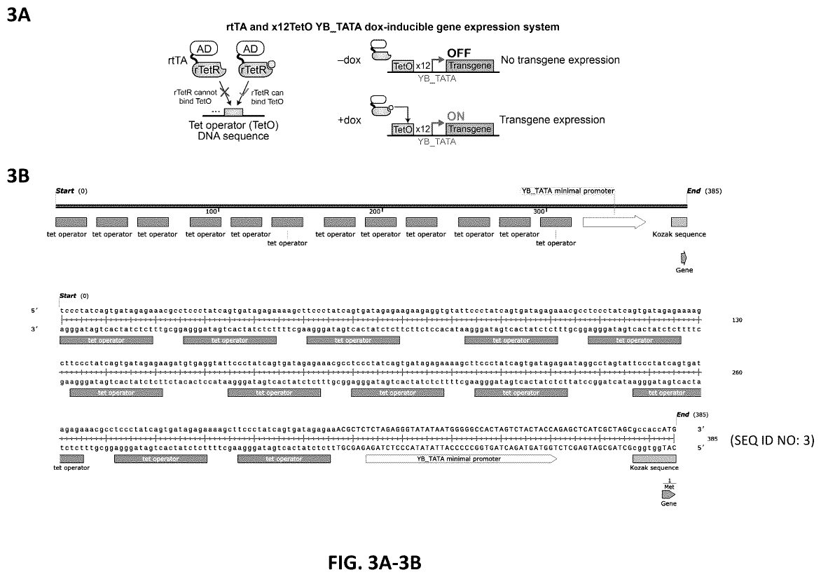

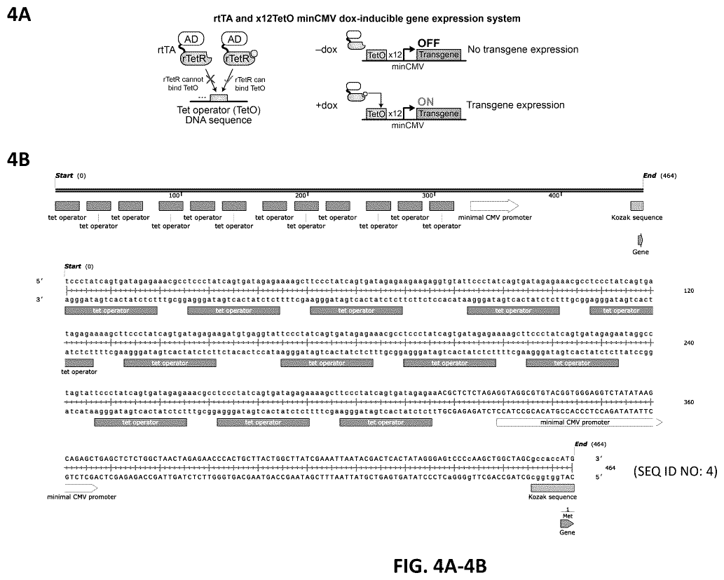

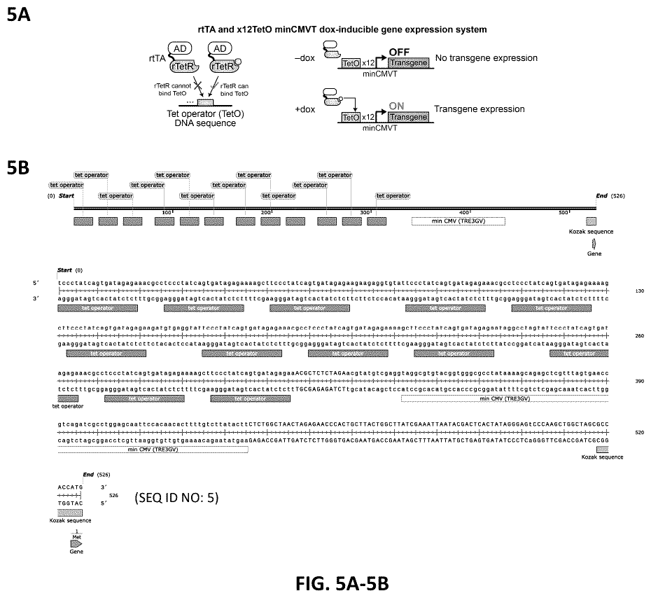

To evaluate target gene expression of x12TetO promoters (FIG. 2A-2B, FIG. 3A-3B, FIG. 4A-4B, FIG. 5A-5B) compared to pTRE3GV (FIG. 1A-1B), HEK293FT cells were transfected with three plasmids for gene expression: a transfection control plasmid that encoded constitutive expression via the human cytomegalovirus (CMV) promoter of mTagBFP2 to identify transfected (blue fluorescence positive) cells; a plasmid that encodes constitutive expression via the human elongation factor-1 alpha (hEF1a) promoter of rtTA; a plasmid that encodes dox-inducible expression of the reporter gene DsRedExpress2 to evaluate promoter activity when cells are treated with dox (FIG. 6A). Reporter expression of each dox-inducible promoter was evaluated as a function of doxycycline (DOX) dose. Data shown in FIG. 6B represent the mean fluorescence intensity (in absolute units of molecules of equivalent PE-TexasRed, MEPTRs) of transfected, single cells. Transfected cells were identified as expressing mTagBFP2. Each data point represents the mean across three biologic replicates and error bars depict the standard error of the mean. FIG. 6C shows background reporter expression of the promoter/reporter alone, background reporter expression of the promoter/reporter and rtTA treated with 0 ng/ml dox, and induced reporter expression of the promoter/reporter and rtTA treated with 1,000 ng/ml dox for each dox-inducible promoter. Data shown represent mean fluorescence intensity of transfected, single cells and is depicted on a logarithmic scale. Each bar represents the mean across three biologic replicates and error bars depict the standard error of the mean.

To further evaluate the dox-inducible x12TetO promoters, HEK293FT-LP cells were transfected with the integration vector and a plasmid encoding expression of Bxb1 recombinase and selected for those that are resistant to both puromycin and blasticidin. Selected cells contained one of the tested dox-inducible promoters regulating expression of an mNeonGreen reporter gene to evaluate promoter activity when cells are treated with dox (FIG. 7B). FIG. 7C shows reporter expression of each dox-inducible promoter when implemented in the landing pad as a function of dox dose. FIG. 7D provides histograms visualizing distribution of reporter expression across treated cells that underly the bulk data shown in FIG. 7C. Exceptionally tight off state of x12TetO YB_TATA (low background expression when treated with 0 ng/ml dox compared to all other tested promoters) was evident. FIG. 7E shows background reporter expression when treated with 0 ng/ml dox and induced reporter expression when treated with 1,000 ng/ml dox for each dox-inducible promoter. Background expression of DoxMax was significantly lower than the other promoter designs (single-factor ANOVA, ***p<0.001). The exceptionally low background of DoxMax facilitated characterization of synTFs in this study by widening the lower end of the range of accessible synTF expression levels in the genome. Additionally, low background has advantages for toxicity and burden caused by even low levels of expression of very strong synTFs. The maximal induced reporter expression when treated with 1,000 ng/ml dox was significantly different between all promoter designs (single-factor ANOVA, p<0.05). Data shown in FIG. 7C-7E represent the fluorescence intensity or mean fluorescence intensity of miRFP720-expressing, single cells. Error bars depict the standard error of the mean (S.E.M.). FIG. 7F shows fold induction of each dox-inducible promoter as a function of dox dose. The x12TetO YB_TATA promoter demonstrated maximal fold induction across the tested promoter variants. Thus, this system was named DoxMax. Data shown represent the fold induction (reporter expression at dose of dox on x-axis divided by the reporter expression in the sample treated with 0 ng/mL dox) of miRFP720-expressing single cells. Each data point represents the mean across three biologic replicates and error bars depict the standard error of the mean. FIG. 7G provides a schematic of all-in-one lentiviral vector for the x12TetO YB TATA-based dox-inducible gene expression system (DoxMax). Briefly, HEK293FT cells were engineered to stably express the cargo depicted here using the depicted lentiviral vector. This workflow involved transducing cells with lentivirus and selecting for cells that were resistant to puromycin. FIG. 7H shows reporter expression of selected cells with a single or multiple copies of this system as a function of dox dose. FIG. 7J shows histograms visualizing distribution of reporter expression across treated cells that underly the bulk data shown in FIG. 7F. FIG. 7J shows background reporter expression when treated with 0 ng/mL dox and induced reporter expression when treated with 100 ng/ml dox. Background reporter expression was not different between populations with single and multicopy integrations of the lentiviral reporter construct (single factor ANOVA, not significant, n.s. p>0.05), suggesting that background expression was still low even when implemented in multiple copies. Although background does not increase with additional copies, induced reporter output at all doxycycline doses greater than 1 ng/ml was significantly increased in cells with multiple copies of the lentiviral reporter construct compared to cells with a single copy (multi-factor ANOVA, **p<0.01). Data shown in

FIG. 7H-7J represent fluorescence intensity or mean fluorescence intensity of mNeonGreen-expressing single cells. Each bar represents the mean across three biologic replicates and error bars depict the standard error of the mean (S.E.M.). FIG. 7K shows fold induction as a function of dox dose. Data shown represent the fold induction (reporter expression at dose of dox on x-axis divided by the reporter expression in the sample treated with 0 ng/ml dox) of mNeonGreen-expressing single cells. Each data point represents the mean across three biologic replicates and error bars depict the standard error of the mean (S.E.M.)

What is claimed is:

1. A method for controlling expression of a target gene in a mammalian cell, comprising:
(a) transiently transfecting or stably genomically integrating a mammalian cell with (1) a first nucleic acid comprising a nucleotide sequence comprising a tetracycline-inducible promoter that controls expression of the target gene; and (2) a second nucleic acid comprising a nucleotide sequence encoding a transcriptional activator that binds to TetO sites, thereby producing an engineered mammalian cell; and
(b) contacting the engineered mammalian cell with a tetracycline to induce expression of the target gene, wherein the tetracycline-inducible promoter comprises a minimal promoter and a TetO site array in which TetO sites are spaced more compactly and with more TetO sites than in pTRE3GV.

2. The method of claim 1, wherein a vector comprises the first and second nucleic acids.

3. The method of claim 1, wherein a first vector comprises the first nucleic acid and a second vector comprises the second nucleic acid.

4. The method of claim 1, wherein each TetO site in the TetO site array are less than 17 base pairs apart.

5. The method of claim 1, wherein each TetO site in the TetO site array are less than 6 base pairs apart.

6. The method of claim 1, wherein the tetracycline is doxycycline.

7. The method of claim 1, wherein the minimal promoter is a variant of a minimal CMV promoter or a synthetic minimal promoter.

8. The method of claim 1, wherein the minimal promoter comprises the nucleotide sequence of SEQ ID NO: 8.

9. The method of claim 7, wherein the first nucleic acid further comprises a GC box upstream from the minimal CMV promoter.

10. The method of claim 1, wherein the tetracycline-inducible promoter exhibits reduced promoter activity in the absence of a tetracycline.

11. The method of claim 1, wherein the tetracycline-inducible promoter provides a higher expression level of a target gene than pTRE3GV.

12. The method of claim 11, wherein the tetracycline-inducible promoter provides a fold induction in expression level of the target gene upon treatment with tetracycline over background expression level that is more than 4-fold higher than that provided by pTRE3GV.

13. The method of claim 1, wherein the TetO site array comprises 12 TetO sites upstream of the minimal promoter.

14. A vector comprising a minimal promoter and a TetO site array in which TetO sites are spaced more compactly and with more TetO sites than in pTRE3GV.

15. The vector of claim 14, wherein each TetO site in the TetO site array are less than 17 base pairs apart.

16. The vector of claim 14, wherein each TetO site in the TetO site array are less than 6 base pairs apart.

17. The vector of claim 14, wherein the minimal promoter is a variant of a minimal CMV promoter or a synthetic minimal promoter.

18. The vector of claim 17, wherein the vector further comprises a GC box upstream from the minimal CMV promoter.

19. The vector of claim 14, wherein the vector exhibits reduced promoter activity in the absence of a tetracycline.

20. The vector of claim 14, wherein the vector provides a higher expression level of a target gene than pTRE3GV.

21. The vector of claim 14, wherein the vector provides a fold induction in expression level of the target gene upon treatment with tetracycline over background expression level that is more than 4-fold higher than that provided by pTRE3GV.

22. The vector of claim 14, wherein the TetO site array comprises 12 TetO sites upstream of the minimal promoter.
